SEQUENCE LISTING (15 sequences)

SEQ ID NO: 1 - DNA, 28 bp, Artificial
  Synthetic Construct
gcatgaattcatgactgccagcgtttcg

SEQ ID NO: 2 - DNA, 36 bp, Artificial
  Synthetic Construct
ggatccccatggtcatttggcgctatttttttatac

SEQ ID NO: 3 - DNA, 39 bp, Artificial
  Synthetic Construct
cgtgaccatgggagtgggatggacaaggattgtgaaatg

SEQ ID NO: 4 - DNA, 33 bp, Artificial
  Synthetic Construct
gcatgggtaccttaagcgtaatctggaacatcg

SEQ ID NO: 5 - DNA, 52 bp, Artificial
  Synthetic Construct
ctcgcccttgctcaccatcccgctgccggacccagcgtaatctggaacatcg

SEQ ID NO: 6 - DNA, 52 bp, Artificial
  Synthetic Construct
cgatgttccagattacgctgggtccggcagcgggatggtgagcaagggcgag

SEQ ID NO: 7 - DNA, 37 bp, Artificial
  Synthetic Construct
ctagctgggtaccgttacttgtacagctcgtccatga

SEQ ID NO: 8 - DNA, 39 bp, Artificial
  Synthetic Construct
cgatacgaattcatggacaaggattgtgaaatgaaacgc

SEQ ID NO: 9 - DNA, 34 bp, Artificial
  Synthetic Construct
ttcatagcggccgcgtcagtttcggccagcaggc

SEQ ID NO: 10 - DNA, 33 bp, Artificial
  Synthetic Construct
gcatgctacgtaatggacaaggattgtgaaatg

SEQ ID NO: 11 - DNA, 32 bp, Artificial
  Synthetic Construct
gagcacttaattaagtttcggccagcaggcgg

SEQ ID NO: 12 - DNA, 48 bp, Artificial
  Synthetic Construct
atcgagctagcgctaccggtcgccaccatggacaaggattgtgaaatg

SEQ ID NO: 13 - DNA, 28 bp, Artificial
  Synthetic Construct
cgtagctcgagagtttcggccagcaggc

SEQ ID NO: 14 - DNA, 32 bp, Artificial
  Synthetic Construct
catgcagatctgagtccggacttgtacagctc

SEQ ID NO: 15 - DNA, 32 bp, Artificial
  Synthetic Construct
ccaggcagatctgtttcggccagcaggcgggg

(19) United States
(12) Patent Application Publication
(10) Pub. No.: US 2006/0292651 A1
(43) Pub. Date: Dec. 28, 2006

(54) Protein labelling with oº-alkylguanine-dna alkyltrnsferase

(76) Inventors: Alexandre Juillerat, Lausanne (CH); Antje Keppler, Lausanne (CH); Kai Johnsson, Lausanne (CH); Thomas Gronemeier, Ecublens (CH); Susanne Gendreizig, Lausanne (CH); Andreas Brecht, Muttenz (CH)

Correspondence Address: WENDEROTH, LIND & PONACK, L.L.P., 2033 K STREET N. W., SUITE 800, WASHINGTON, DC, 20006-1021, US

(21) Appl. No.: 10/529,647

(22) PCT Filed: Oct. 1, 2003
(86) PCT No.: PCT/EP03/10859

§ 371 Date: Apr. 29, 2005

(30) Foreign Application Priority Data
Oct. 3, 2002 (EP) 02405855.4

(51) Int. Cl. C12Q 1/48; C12N 9/12

(52) U.S. Cl. 435/15; 435/194

(57) ABSTRACT

The present invention relates to methods of transferring a label from suitable substrates to O6-alkylguanine-DNA alkyltransferase (AGT) fusion proteins, to suitable fusion proteins, to suitable variants of AGT, and to novel labelled fusion proteins obtained. A protein of interest is incorporated into an AGT fusion protein, the AGT fusion protein is contacted with an AGT substrate carrying a label, and the AGT fusion protein is detected and/or manipulated using the label in a system designed for recognising and/or handling the label.

### FIELD OF THE INVENTION

The present invention relates to methods of transferring a label from suitable substrates to O⁶-alkylguanine-DNA alkyltransferase fusion proteins, and to novel labelled fusion proteins obtained.

### BACKGROUND OF THE INVENTION

The mutagenic and carcinogenic effects of electrophiles such as N-methyl-N-nitrosourea are mainly due to the O⁶-alkylation of guanine in DNA. To protect themselves against DNA-alkylation, mammals and bacteria possess a protein, O⁶-alkylguanine-DNA alkyltransferase (AGT) which repairs these lesions. AGT transfers the alkyl group from the position O-6 of alkylated guanine and guanine derivatives to the mercapto group of one of its own cysteines, resulting in an irreversibly alkylated AGT. The underlying mechanism is a nucleophilic reaction of the S_{N}2 type which explains why not only methyl groups, but also benzylic groups are easily transferred. As overexpression of AGT in tumour cells is the main reason for resistance to alkylating drugs such as procarbazine, dacarbazine, temozolomide and bis-2-chloroethyl-N-nitrosourea, inhibitors of AGT have been proposed for use as sensitisers in chemotherapy (Pegg et al., Prog Nucleic Acid Res Mol Biol 51: 167-223,1995).

DE 199 03 895 discloses an assay for measuring levels of AGT which relies on the reaction between biotinylated O⁶-alkylguanine derivatives and AGT which leads to biotinylation of the AGT. This in turn allows the separation of the AGT on a streptavidin coated plate and its detection, e.g. in an ELISA assay. The assay is suggested for monitoring the level of AGT in tumour tissue and for use in screening for AGT inhibitors.

Damoiseaux et al., ChemBiochem. 4: 285287, 2001, disclose modified O⁶-alkylated guanine derivatives incorporated into oligodeoxyribonucleotides for use as chemical probes for labelling AGT, again to facilitate detecting the levels of this enzyme in cancer cells to aid in research and in chemotherapy.

PCT/GB02/01636 discloses a method for detecting and/or manipulating a protein of interest wherein the protein is fused to AGT and the AGT fusion protein contacted with an AGT substrate carrying a label, and the AGT fusion protein detected and optionally further manipulated using the label. Several AGT fusion proteins to be used, general structural principles of the AGT substrate and a broad variety of labels and methods to detect the label useful in the method are described.

### SUMMARY OF THE INVENTION

The invention relates to a method for detecting and/or manipulating a protein of interest, wherein the protein of interest is incorporated into an AGT fusion protein, the AGT fusion protein is contacted with a suitable AGT substrate carrying a label, and the AGT fusion protein is detected or manipulated or both manipulated and detected in any order using the label in a system designed for recognising and/or handling the label.

The protein of interest according to the invention is selected from the group consisting of enzymes, DNA-binding proteins, transcription regulating proteins, membrane proteins, nuclear receptor proteins, nuclear localization signal proteins, protein cofactors, small monomeric GTPases, ATP-binding cassette proteins, intracellular structural proteins, proteins with sequences responsible for targeting proteins to particular cellular compartments, proteins generally used as labels or affinity tags, and domains or subdomains of the aforementioned proteins, excluding the major head protein D of phage λ (gpD) and those particular proteins of interest disclosed in PCT/GB02/01636 (WO 02/083937).

The AGT fusion protein may consist of one or more, e.g. one, two or three, proteins of interest fused to AGT at the N—, C or N— and C-terminal of AGT. AGT may be human AGT (hAGT), other mammalian AGT, or a variant of a wild-type AGT with one or more amino acid substitution, deletion or addition.

The invention relates also to the novel AGT fusion proteins as such, and in particular to labelled AGT fusion proteins obtained in the method of the invention comprising an AGT fusion protein covalently bound to a substrate carrying a label.

### DETAILED DESCRIPTION OF THE INVENTION

In the present invention a protein or peptide of interest is fused to an O⁶-alkylguanine-DNA alkyltransferase (AGT). The protein or peptide of interest may be of any length and both with and without secondary, tertiary or quatemary structure, and preferably consists of at least twelve amino acids and up to 2000 amino acids, preferably between 50 and 1000 amino acids.

The protein of interest according to the invention is selected from the group consisting of enzymes, e.g.

transferases (EC 2), more specific a transferase transferring an alkyl or aryl group other than a methyl group (EC 2.5), in particular a glutathione transferase (EC 2.5.1.18), or a kinase, that is a transferase transferring phosphorus containing groups (EC 2.7), in particular a kinase with an alcohol group as acceptor (EC 2.7.1), such as a protein kinase with serine and threonine as the phosphorylated target sites in the substrate protein, e.g. casein-kinase from yeast (EC 2.7.1.37), or a tyrosine protein kinase (EC 2.7.1.112);

or e.g. oxidoreductases (EC 1), more specific an oxidoreductase acting on peroxide as acceptor (EC 1.11), in particular the enzyme cytochrome C peroxidase (EC 1.11.1.5); or e.g. hydrolases (EC 3), more specific a hydrolase acting on an ester bond (EC 3.1), in particular a phosphoric monoester hydrolase (EC 3.1.3), such as a protein phosphoric monoester hydrolase; or a hydrolase hydrolyzing peptide bonds, also known as peptidase or protease (EC 3.4), in particular a caspase;

DNA-binding proteins, more specific transcription repressor proteins which are protein factors inhibiting mRNA synthesis, specifically a protein factor inhibiting mRNA synthesis in E. coli, in particular the DNA-binding domain of the LexA protein;

transcription regulating proteins, more specific transcription repressor proteins, in particular transcription repressor proteins containing a tryptophan/aspartate repeat structure, specifically the S. cerevisiae transcription repressor Tup1;

membrane proteins, e.g. membrane proteins showing at least one transmembrane helix, more specific membrane proteins from the endoplasmatic reticulum (ER) membrane, in particular membrane proteins being active in protein translocation into the ER, such as the ER transmembrane protein Sec62;

or e.g. a protein from the family of 7-transmembrane helix (7-TM) proteins, more specific a 7-TM protein being a G-protein coupled receptor (GPCR), in particular those that bind macromolecular ligands with a molecular weight above 1 kDa, such as a mammalian, e.g. human, neurokinin-1-receptor (NK1);

or e.g. transmembrane ion channel proteins from the cell membrane, in particular ligand gated ion channel proteins, more specific a ligand gated ion channel protein sensitive to serotonin, such as the serotonin receptor 5-HT3;

or e.g. membrane receptors other than ion channels and G-protein coupled receptors;

or e.g. peroxisomal membrane proteins, in particular from yeast, such as the protein Pex15;

nuclear receptor proteins, e.g. nuclear receptor proteins from the family of transcription factors, more specific nuclear receptor proteins from the family of ligand inducible transcription factors, in particular a nuclear receptor from the family of steroid, e.g. estrogen, receptors, such as the human estrogen receptor hER;

nuclear localization signal proteins, such as the nuclear localization signal from the Simian Virus 40 (SV40);

protein cofactors, e.g. proteins containing an ubiquitin sequence in their genetic structure;

small monomeric GTPases, more specific membrane-adherent small monomeric GTPases, e.g. a member of the Ras family,

ATP-binding cassette (ABC) proteins, e.g. a multiple drug resistance protein;

intracellular structural proteins, more specifically proteins of the cytoskeleton, more specifically human cytoplasmic β-actin;

proteins with sequences responsible for targeting proteins to particular cellular compartments, e.g. to the Golgi apparatus, the endoplasmatic reticulum (ER), the mitochondria, the plasma membrane or the peroxisome;

proteins generally used as labels or affinity tags, e.g. fluorescent proteins giving a fluorescent signal on excitation with UV or visible radiation, in particular fluorescent proteins from the family known as green fluorescent proteins (GFP), such as the fluorescent protein known as enhanced cyano fluorescent protein (ECFP);

and domains or subdomains of the aforementioned proteins.

Furthermore, the protein of interest according to the invention is selected according to source. In particular, proteins of interest are those present in bacterial species, e.g. salmonella, more specific salmonella typhi or salmonella typhimurium, mycobacteria, more specific mycobacterium tuberculensis, or staphylococci, more specific staphylococcus aureus, or from a viral source, e.g. human immunodeficiency virus (HIV), human influenza virus, or hepatitis virus.

Preferred groups of proteins of interest are, for example,

receptors, e.g. membrane receptors, in particular 7-TM receptors (GPCRs), receptors with enzymatic activity, in particular of a kinase type which might require dimerization to be active, ion channels, and membrane proteins involved in-virus docking and virus entering cells, or e.g. intracellular receptors, in particular receptors for compounds crossing the membrane, such as receptors for steroid hormones;

extracellular signaling molecules and signaling factors, e.g. interleukins, growth factors, releasing hormones, prostaglandins, insulin and glucagon;

proteins of intracellular signal cascades, e.g. enzymes and cofactors involved in phosphatidinyl-inositol signaling, and in cAMP and cGMP generation, membrane adherent and free kinases, kinase-kinases as well as phosphatases, and the terminally activated or deactivated enzymes of intracellular signaling cascades, in particular those activating caspases;

hormones, and enzymes involved in the synthesis, liberation, activation, receptor activity and desactivation of hormones;

membrane surface markers correlating with the cell status, e.g. alpha-fetoprotein;

and proteins involved in blood pressure control and-heart function, e.g. ACE inhibitors, kidney receptors and kidney channel proteins, and cardiac potassium channel proteins.

Excluded from the scope of the claims of the present invention are fusion proteins with the major head protein D of phage λ (gpD), and with protein of interest disclosed in PCT/GB02101636 (WO 02/083937), in particular MHHHHHHSSA-hAGT, the fusion protein of the short peptide His₆ further comprising methionine (M), serine (S) and alanine (A), hAGT-DHFR-HA, the fusion protein of hAGT, a short linker peptide, dihydrofolate reductase from mouse and the Ha epitope; V5-NLS-B42-hAGT, the fusion protein of the V5 epitope, the SV40 large T antigen nuclear localization sequence, the artificial transcriptional activator B42, a linker peptide and hAGT; hAGT-HA-Ura3, the fusion protein of hAGT, the Ha epitope and the yeast enzyme orotic acid decarboxylase Ura3; and hAGT-SSN6, the fusion protein of hAGT, a short linker peptide and a yeast repressor of DNA transcription named SSN6.

Disclosed are fusion proteins made from wild-type human AGT (hAGT), other mammalian AGT, e.g. rat or mouse AGT, or variants of such AGT DNA on the one side and proteins of interest (as listed above) encoding sequences either attached to the N-terminal (N) or the C-terminal (C) side or N— and C-terminal side of the AGT DNA sequence, leading to the fusion proteins of the invention. Fusion proteins may further contain suitable linkers, e.g. linkers which may be susceptible to enzyme cleavage under suitable conditions, between AGT and the protein of interest and/or between two proteins of interest in a fusion protein. Examples of such linkers are those which are cleavable at the DNA stage by suitable restriction enzymes, e.g. AGATCT cleavable by BgI II, and/or linkers cleavable by suitable enzymes at the protein stage, e.g. tobacco etch virus NIa (TEV) protease.

Fusion proteins may be expressed in prokaryotic hosts, preferably E coli, or eukaryotic hosts, e.g. eubacteria, yeast, insect cells or mammalian cells.

The O⁶-alkylguanine-DNA alkyltransferase (AGT) has the property of transferring a label present on a substrate to one of the cysteine residues of the AGT forming part of a fusion protein. In preferred embodiments, the AGT is a known human O⁶-alkylguanine-DNA alkyltransferase, hAGT. Murine or rat forms of the enzyme are also considered provided they have similar properties in reacting with a substrate like human AGT. In the present invention, O⁶-alkylguanine-DNA alkyltransferase also includes variants of a wild-type AGT which may differ by virtue of one or more, e.g. one, two, three or four, amino acid substitutions, deletions or additions, but which still retain the property of transferring a label present on a substrate to the AGT part of the fusion protein. AGT variants may be obtained by chemical modification using techniques well known to those skilled in the art. AGT variants may preferably be produced using protein engineering techniques known to the skilled person and/or using molecular evolution to generate and select new O⁶-alkylguanine-DNA alkyltransferases. Such techniques are e.g. saturation mutagenesis, error prone PCR to introduce variations anywhere in the sequence, DNA shuffling used after saturation mutagenesis and/or error prone PCR, or family shuffling using genes from several species.

With the aid of the phage display method mutants are found with significantly increased activity towards O⁶-benzylguanine and AGT substrates of the invention. hAGT can be functionally displayed as a fusion protein with the major head protein D on phage λ, and the unusual mechanism of hAGT can be used to select phage λ displaying hAGT out of mixtures of wild-type phage λ (Damoiseaux et al., ChemBiochem. 4: 285-287, 2001). hAGT may also be functionally displayed on filamentous phage as a fusion protein with the phage capsid protein pill.

In the structure of hAGT bound with O⁶-benzylguanine in its active site, four amino acids are in proximity of either the benzyl ring (Pro140, Ser159, Gly160), or could make contact with the N9 of the nucleobase (Asn157). Mutations at position Pro140 and Gly160 have previously been shown to affect the reaction of hAGT with O⁶-benzylguanine (Xu-Welliver et al., Biochemical Pharmacology 58: 1279-85, 1999): A proline at position 140 is believed to be essential for its interaction with the benzyl ring, and the mutation Gly160Trp has been shown to increase the reactivity of hAGT towards O⁶-benzylguanine. Particular variants considered in this invention are those with Phe or Met in position 140; Gly, Pro, Arg or Trp at position 157, in particular Gly; Glu, Asn, Pro or Gln at position 159, in particular Glu; and Ala, Trp, Cys or Val at position 160, in particular Trp. The preferred variants are the one wherein Asn¹⁵⁷ is replaced by Gly and Ser¹⁵⁹ by Glu, and the one wherein Gly¹⁶⁰ is replaced by Ala or Trp. Most preferred is the variant wherein Asn¹⁵⁷ is replaced by Ser, Ser¹⁵⁹ by His, and Gly¹⁶⁰ by Asn.

The fusion protein comprising protein of interest and an O⁶-alkylguanine-DNA alkyl-transferase (AGT) is contacted with a particular substrate having a label. Conditions of reaction are selected such that the AGT reacts with the substrate and transfers the label of the substrate. Usual conditions are a buffer solution at around pH 7 at room temperature, e.g. around 25° C. However, it is understood that AGT reacts also under a variety of other conditions, and those conditions mentioned here are not limiting the scope of the invention.

AGT irreversibly transfers the alkyl group from its substrate, O⁶-alkylguanine-DNA, to one of its cysteine residues. A substrate analogue that rapidly reacts with hAGT is O⁶-benzyl-guanine, the second order rate constant being approximately 10³ sec⁻¹ M⁻¹. Substitutions of O⁶-benzylguanine at the C4 of the benzyl ring do not significantly affect the reactivity of hAGT against O⁶-benzylguanine derivatives, and this property has been used to transfer a label attached to the C4 of the benzyl ring to AGT.

The label part of the substrate can be chosen by those skilled in the art dependent on the application for which the fusion protein is intended. After contacting the fusion protein comprising AGT with the substrate, the label is covalently bonded to the fusion protein. The labelled AGT fusion protein is then further manipulated and/or detected by virtue of the transferred label.

Under “manipulation” any physical or chemical treatment is understood. For instance manipulation may mean isolation from cells, purification with standard purification techniques, e.g. chromatography, reaction with chemical reagents or with the binding partner of a binding pair, in particular if the binding partner is fixed to a solid phase, and the like. Such manipulation may be dependent on the label L, and may occur in addition to “detection” of the labelled fusion protein. If the labelled fusion protein is both manipulated and detected, detection may be before or after manipulation, or may occur during manipulation as defined herein.

The particular AGT substrates are compounds of the formula 1

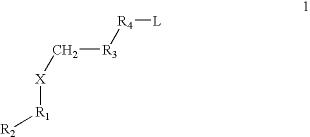

wherein R₁—R₂ is a group recognized by AGT as a substrate;

X is oxygen or sulfur;

R₃ is an aromatic or a heteroaromatic group, or an optionally substituted unsaturated alkyl, cycloalkyl or heterocyclyl group with the double bond connected to CH₂;

R₄ is a linker; and

L is a label, a plurality of same or different labels, a bond connecting R₄ to R₁ forming a cyclic substrate, or a further group —R₃—CH,—X—R₁—R_{2.}

In a group R₁—R₂, the residue R₁ is preferably a heteroaromatic group containing 1 to 5 nitrogen atoms, recognized by AGT as a substrate, preferably a purine radical of the formula 2

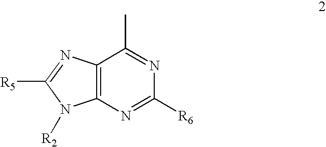

wherein R₂ is hydrogen, alkyl of 1 to 10 carbon atoms, or a saccharide moiety;

R₅ is hydrogen, halogen, e.g. chloro or bromo, trifluoromethyl, or hydroxy; and

R₆ is hydrogen, hydroxy or unsubstituted or substituted amino.

If R₅ or R₆ is hydroxy, the purine radical is predominantly present in its tautomeric form wherein a nitrogen adjacent to the carbon atom bearing R₅ or R₆ carries a hydrogen atom, the double bond between this nitrogen atom and the carbon atom bearing R₅ or R₆ is a single bond, and R₅ or R₆ is double bonded oxygen, respectively.

A substituted amino group R₆ is lower alkylamino of 1 to 4 carbon atoms or acylamino, wherein the acyl group is lower alkylcarbonyl with 1 to 5 carbon atoms, e.g. acetyl, propionyl, n- or isopropylcarbonyl, or n-, iso- or tert-butylcarbonyl, or arylcarbonyl, e.g. benzoyl.

If R₆ is unsubstituted or substituted amino and the residue X connected to the bond of the purine radical is oxygen, the residue of formula 2 is a guanine derivative.

A saccharide moiety R₂ is a saccharide monomer or oligomer connected with a spacer of variable length to the N⁹ position of the guanine base. The spacer in this context is an alkyl chain preferably from 1 to 15 carbon atoms, a polyethylene glycol spacer consisting of 1 to 200 ethylene glycol units, an amide group —CO—NH—, an ester group CO—O—, an alkylene group —CH═CH— or a combination of alkyl chain, polyethylene glycol group, amide group, ester group, and alkylene group.

In the context of this invention, a saccharide moiety R₂ further includes a β-D-2′-deoxyribosyl, or a β-D-2′-deoxyribosyl being incorporated into a single stranded oligodeoxyribonucleotide having a length of 2 to 99 nucleotides, wherein the guanine derivative R₁ occupies any position within the oligonucleotide sequence.

In another preferred embodiment of the invention the group R₁—R₂ is a 8-azapurine radical, wherein the moiety C—R₅ of the radical of formula 2 is replaced by nitrogen, and R₂ and R₆ have the meaning as defined under formula 2.

X is preferably oxygen.

R₃ as an aromatic or a heteroaromatic group, or an optionally substituted unsaturated alkyl, cycloalkyl or heterocyclyl group is a group sterically and electronically accepted by AGT (in accordance with its reaction mechanism) which allows the covalent transfer of the R₃—R₄-L unit to the fusion protein. In a R₃—R₄-L unit, R₄-L may also have the meaning of a plurality of same or different linkers R₄ carrying a plurality of same or different labels L.

R₃ as an aromatic group is preferably phenyl or naphthyl, in particular phenyl, e.g. phenyl substituted by R₄ in para or meta position.

A heteroaromatic group R₃ is a mono- or bicyclic heteroaryl group comprising zero, one, two, three or four ring nitrogen atoms and zero or one oxygen atom and zero or one sulfur atom, with the proviso that at least one ring carbon atom is replaced by a nitrogen, oxygen or sulfur atom, and which has 5 to 12, preferably 5 or 6 ring atoms; and which in addition to carrying a substituent R₄ may be unsubstituted or substituted by one or more, especially one, further substituent selected from the group consisting of lower alkyl, such as methyl, lower alkoxy, such as methoxy or ethoxy, halogen, e.g. chlorine, bromine or fluorine, halogenated lower alkyl, such as trifluoromethyl, or hydroxy. Preferably the heteroaryl group R₃ is triazolyl, especially 1-triazolyl, carrying the further substituent R₄ in the 4- or 5-position, tetrazolyl, especially 1-tetrazolyl, carrying the further substituent R₄ in the 4- or 5-position or 2-tetrazolyl carrying the further substituent in 5 position, isoxazolyl, especially 3-isoxazolyl carrying the further substituent in 5 position, or 5-isoxazolyl, carrying the further substituent in 3 position, or thienyl, especially 2-thienyl, carrying the further substituent R₄ in 3-, 4- or 5-position, preferably 4- position, or 3-thienyl, carrying the further substituent R₄ in 4-position.

An optionally substituted unsaturated alkyl group R₃ is 1-alkenyl carrying the further substituent R₄ in 1 or 2 position, preferably in 2 position, or 1-alkynyl. Substituents considered in 1-alkenyl are e.g. lower alkyl, e.g. methyl, lower alkoxy, e.g. methoxy, lower acyloxy, e.g. acetoxy, or halogenyl, e.g. chloro.

An optionally substituted unsaturated cycloalkyl group is a cycloalkyl group with 3 to 7 carbon atoms unsaturated in 1 position, e.g. 1-cyclopentyl or 1-cyclohexyl, carrying the further substituent R₄ in any position. Substituents considered are e.g. lower alkyl, e.g. methyl, lower alkoxy, e.g. methoxy, lower acyloxy, e.g. acetoxy, or halogenyl, e.g. chloro.

An optionally substituted unsaturated heterocyclyl group has 3 to 12 atoms, 1 to 5 heteroatoms selected from nitrogen, oxygen and sulfur, and a double bond in the position connecting the heterocyclyl group to methylene CH₂. Substituents considered are e.g. lower alkyl, e.g. methyl, lower alkoxy, e.g. methoxy, lower acyloxy, e.g. acetoxy, or halogenyl, e.g. chloro. In particular, an optionally substituted unsaturated heterocyclyl group is a partially saturated heteroaromatic group as defined hereinbefore for a heteroaromatic group R₃. An example of such a heterocyclyl group is isoxazolidinyl, especially 3-isoxazolidinyl carrying the further substituent in 5 position, or 5-isoxazolidinyl, carrying the further substituent in 3 position.

A linker group R₄ is preferably a flexible linker connecting a label L or a plurality of same or different labels L to the substrate. Linker units are chosen in the context of the envisioned application, i.e. in the transfer of the substrate to a fusion protein comprising AGT. They also increase the solubility of the substrate in the appropriate solvent. The linkers used are chemically stable under the conditions of the actual application. The linker does not interfere with the reaction with AGT nor with the detection of the label L, but may be constructed such as to be cleaved at some point in time after the reaction of the compound of formula 1 with the fusion protein comprising AGT.

A linker R₄ is a straight or branched chain alkylene group with 1 to 300 carbon atoms, wherein optionally

(a) one or more carbon atoms are replaced by oxygen, in particular wherein every third carbon atom is replaced by oxygen, e.g. a poylethyleneoxy group with 1 to 100 ethyleneoxy units;

(b) one or more carbon atoms are replaced by nitrogen carrying a hydrogen atom, and the adjacent carbon atoms are substituted by oxo, representing an amide function —NH—CO;

(c) one or more carbon atoms are replaced by oxygen, and the adjacent carbon atoms are substituted by oxo, representing an ester function —O—CO—;

(d) the bond between two adjacent carbon atoms is a double or a triple bond, representing a function —CH═CH— or —C≡C—;

(e) one or more carbon atoms are replaced by a phenylene, a saturated or unsaturated cycloalkylene, a saturated or unsaturated bicycloalkylene, a bridging heteroaromatic or a bridging saturated or unsaturated heterocyclyl group;

(f) two adjacent carbon atoms are replaced by a disulfide linkage —S—S—;

or a combination of two or more, especially two or three, alkylene and/or modified alkylene groups as defined under (a) to (f) hereinbefore, optionally containing substituents.

Substituents considered are e.g. lower alkyl, e.g. methyl, lower alkoxy, e.g. methoxy, lower acyloxy, e.g. acetoxy, or halogenyl, e.g. chloro.

Further substituents considered are e.g. those obtained when an α-amino acid, in particular a naturally occurring α-amino acid, is incorporated in the linker R₄ wherein carbon atoms are replaced by amide functions —NH—CO— as defined under (b). In such a linker part of the carbon chain of the alkylene group R₄ is replaced by a group —NH—CHR—CO)ₙ— wherein n is between 1 and 100 and R represents a varying residue of an α-amino acid.

A further substituent is one which leads to a photocleavable linker R₄, e.g. an o-nitrophenyl group. In particular this substituent o-nitrophenyl is located at a carbon atom adjacent to an amide bond, e.g. in a group —NH—CO—CH₂CH(o-nitrophenyl)—NH—CO—, or as a substituent in a polyethylene glycol chain, e.g. in a group —O—CH₂—CH(o-nitrophenyl)-O—. Other photocleavable linkers considered are, e.g. phenacyl, alkoxybenzoin, benzylthioether and pivaloyl glycol derivatives.

A phenylene group replacing carbon atoms as defined under (e) hereinbefore is e.g. 1,2-,1,3-, or preferably 1,4-phenylene. A saturated or unsaturated cycloalkylene group replacing carbon atoms as defined under (e) hereinbefore is e.g. cyclopentylene or cyclohexylene, or also cyclohexylene being unsaturated e.g. in 1- or in 2-position. A saturated or unsaturated bicycloalkylene group replacing carbon atoms as defined under (e) hereinbefore is e.g. bicyclo[2.2.1]heptylene or bicyclo[2.2.2]octylene, optionally unsaturated in 2-position or doubly unsaturated in 2- and 5-position. A heteroaromatic group replacing carbon atoms as defined under (e) hereinbefore is e.g. triazolidene, preferably 1,4-triazolidene, or isoxazolidene, preferably 3,5-isoxazolidene. A saturated or unsaturated heterocyclyl group replacing carbon atoms as defined under (e) hereinbefore is e.g. 2,5- tetrahydrofuranediyl or 2,5-dioxanediyl, or isoxazolidinene, preferably 3,5-isoxazolidinene. A particular heterocyclyl group considered is a saccharide moiety, e.g. an α- or β-furanosyl or α- or β-pyranosyl moiety.

A linker R₄ may carry one or more same or different labels, e.g. 1 to 100 same or different labels, in particular 1 to 5, preferably one, two or three, in particular one or two same or different labels.

The label part L of the substrate can be chosen by those skilled in the art dependent on the application for which the fusion protein is intended. Labels may be e.g. such that the labelled fusion protein is easily detected or separated from its environment. Other labels considered are those which are capable of sensing and inducing changes in the environment of the labelled fusion protein and/or labels which aid in manipulating the fusion protein by the physical and/or chemical properties specifically introduced by the label to the fusion protein.

Examples of labels L include a spectroscopic probe such as a fluorophore, a chromophore, a magnetic probe or a contrast reagent; a radioactively labelled molecule; a molecule which is one part of a specific binding pair which is capable of specifically binding to a partner; a molecule that is suspected to interact with other biomolecules; a library of molecules that are suspected to interact with other biomolecules; a molecule which is capable of crosslinking to other molecules; a molecule which is capable of generating hydroxyl radicals upon exposure to H₂O₂ and ascorbate, such as a tethered metal-chelate; a molecule which is capable of generating reactive radicals upon irradiation with light, such as malachite green; a molecule covalently attached to a solid support, where the support may be a glass slide, a microtiter plate or any polymer known to those proficient in the art; a nucleic acid or a derivative thereof capable of undergoing base-pairing with its complementary strand; a lipid or other hydrophobic molecule with membrane-inserting properties; a biomolecule with desirable enzymatic, chemical or physical properties; or a molecule possessing a combination of any of the properties listed above.

When the label L is a fluorophore, a chromophore, a magnetic label, a radioactive label or the like, detection is by standard means adapted to the label and whether the method is used in vitro or in vivo. The method can be compared to the applications of the green fluorescent protein (GFP) which is genetically fused to a protein of interest and allows protein investigation in the living cell. Particular examples of labels L are also boron compounds displaying non-linear optical properties, or a member of a FRET pair which changes its spectroscopic properties on reaction of the labelled substrate with the AGT fusion protein.

Depending on the properties of the label L, the fusion protein comprising protein of interest and AGT may be bound to a solid support. The label of the substrate reacting with the fusion protein comprising AGT may already be attached to a solid support when entering into reaction with AGT, or may subsequently, i.e. after transfer to. AGT, be used to attach the AGT fusion protein to a solid support. The label may be one member of a specific binding pair, the other member of which is attached or attachable to the solid support, either covalently or by any other means. A specific binding pair considered is e.g. biotin and avidin or streptavidin. Either member of the binding pair may be the label L of the substrate, the other being attached to the solid support. Further examples of labels allowing convenient binding to a solid support are e.g. maltose binding protein, glycoproteins, FLAG tags, or reactive substituents allowing chemoselective reaction between such substituent with a complementary functional group on the surface of the solid support. Examples of such pairs of reactive substituents and complementary functional group are e.g. amine and activated carboxy group forming an amide, azide and a propiolic acid derivative undergoing a 1,3-dipolar cycloaddition reaction, amine and another amine functional group reacting with an added bifunctional linker reagent of the type of activated bis-dicarboxylic acid derivative giving rise to two amide bonds, or other combinations known in the art.

Examples of a convenient solid support are e.g. chemically modified oxidic surfaces, e.g. silicon dioxide, tantalum pentoxide, titanium dioxide, glass surfaces, e.g. glass slides, polymer surfaces, e.g. microtiter plates, in particular functionalised polymers (e.g. in the form of beads), or also chemically modified metal surfaces, e.g. noble metal surfaces such as gold or silver surfaces, and suitable sensor elements made of any of the aforementioned materials. Irreversibly attaching and/or spotting AGT substrates may then be used to attach AGT fusion proteins in a spatially resolved manner, particularly through spotting, on the solid support representing protein microarrays, DNA microarrays or arrays of small molecules.

When the label L is capable of generating reactive radicals, such as hydroxyl radicals, upon exposure to an external stimulus, the generated radicals can then inactivate the AGT fusion proteins as well as those proteins that are in close proximity of the AGT fusion protein, allowing to study the role of these proteins. Examples of such labels are tethered metal-chelate complexes that produce hydroxyl radicals upon exposure to H₂O₂ and ascorbate, and chromophores such as malachite green that produce hydroxyl radicals upon laser irradiation. The use of chromophores and lasers to generate hydroxyl radicals is also known in the art as chromophore assisted laser induced inactivation (CALI). In the present invention, labelling AGT fusion proteins with chromophores such as malachite green and subsequent laser irradiation inactivates the AGT fusion protein as well as those proteins that interact with the AGT fusion protein in a time-controlled and spatially-resolved manner. This method can be applied both in vivo or in vitro. Furthermore, proteins which are in close proximity of the AGT fusion protein can be identified as such by either detecting fragments of that protein by a specific antibody, by the disappearance of those proteins on a high-resolution 2D-electrophoresis gels or by identification of the cleaved protein fragments via separation and sequencing techniques such as mass spectrometry or protein sequencing by N-terminal degradation.

When the label L is a molecule that can cross-link to other proteins, e.g. a molecule containing functional groups such as maleimides, active esters or azides and others known to those proficient in the art, contacting such labelled AGT substrates with AGT fusion proteins that interact with other proteins (in vivo or in vitro) leads to the covalent cross-linking of the AGT fusion protein with its interacting protein via the label. This allows the identification of the protein interacting with the AGT fusion protein. Labels L for photo cross-linking are e.g. benzophenones. In a special aspect of cross-linking the label L is a molecule which is itself an AGT substrate leading to dimerization of the AGT fusion protein. The chemical structure of such dimers may be either symmetrical (homodimers) or unsymmetrical (heterodimers).

Other labels L considered are for example fullerenes, boranes for neutron capture treatment, nucleofides or oligonucleotides, e.g. for self-addressing chips, peptide nucleic acids, and metal chelates, e.g. platinum chelates that bind specifically to DNA.

If the substrate carries two or more labels, these labels may be identical or different.

The present invention provides a method to label AGT fusion proteins both in vivo as well as in vitro. The term in vivo labelling of a AGT fusion protein includes labelling in all compartments of a cell as well as of AGT fusion proteins pointing to the extracellular space. If the labelling of the AGT fusion protein is done in vivo and the protein fused to the AGT is a membrane protein, more specifically a plasma membrane protein, the AGT part of the fusion protein can be attached to either side of the membrane, e.g. attached to the cytoplasmic or the extracellular side of the plasma membrane.

If the labelling is done in vitro, the labelling of the fusion protein can be either performed in cell extracts or with purified or enriched forms of the AGT fusion protein.

If the labelling is done in vivo or in cell extracts, the labelling of the endogenous AGT of the host is advantageously taken into account. If the endogenous AGT of the host does not accept O⁶-alkylguanine derivatives or related compounds as a substrate, the labelling of the fusion protein is specific. In mammalian cells, e.g. in human, murine, or rat cells, labelling of endogenous AGT is possible. In those experiments where the simultaneous labelling of the endogenous AGT as well as of the AGT fusion protein poses a problem, known AGT-deficient cell lines can be used.

In a particular aspect, the present invention provides a method of determining the interaction of a candidate compound or library of candidate compounds with a target protein or library of target proteins. Examples of candidate compounds and target proteins include ligands and proteins, drugs and targets of the drug, or small molecules and proteins. In this particular method of the invention, the protein of interest fused to the AGT comprises a DNA binding domain of a transcription factor or an activation domain of a transcription factor. The putative protein target of the substances or library of proteins is linked to either of the. DNA binding domain or the activation domain of the transcription factor in a way that a functional transcription factor can be formed, and the label L of the AGT substrate according to the invention is a candidate compound or library of candidate compounds suspected of interacting with the target substance or substances. The candidate compound or library of candidate compounds being part of the substrate is then transferred to the AGT fusion protein. On transfer the AGT fusion protein(s) comprising the target substance(s) now are labelled with the candidate compound(s). The interaction of a candidate compound joined to the AGT fusion protein with the target protein fused to either the DNA binding domain or the activation domain leads to the formation of a functional transcription factor. The activated transcription factor can then drive the expression of a reporter which, if the method is carried out in cells, can be detected if the expression of the reporter confers a selective advantage on the cells. In particular embodiments, the method may involve one or more further steps such as detecting, isolating, identifying or characterising the candidate compound(s) or target substance(s).

In a specific example the label L is a drug or a biological active small molecule that binds to a yet unidentified protein Y. A cDNA library of the organism which is expected to express the unknown target protein Y is fused to the activation domain of a transcription factor, and the AGT is fused to the DNA binding domain of a transcription factor, or alternatively, the cDNA library expected to express the unknown target protein Y is fused to the DNA binding domain of a transcription factor, and the AGT is fused to the activation domain of a transcription factor. Adding the AGT substrate of the invention comprising such a label L leads to the formation of a functional transcription factor and gene expression only in the case where this molecule binds to its target protein Y present in the cDNA library and fused to the activation-domain or binding domain, respectively. If gene expression is coupled to a selective advantage, the corresponding host carrying the plasmid with the gene coding for the target protein Y of the drug or bioactive molecule can be identified.

In a further specific example the label L is a library of chemical molecules. The library is expected to contain yet unidentified compounds that bind to a known drug target protein Y under in vivo conditions. The target protein Y is fused to the activation domain of a transcription factor and the AGT is fused to the DNA binding domain of a transcription factor, or alternatively, the target protein Y is fused to the DNA binding domain of a transcription factor and the AGT is fused to the activation domain of a transcription factor. Adding the substrate carrying the library of chemical compounds will lead to the covalent attachment of the chemical compounds of the library to the AGT, which is fused to either the DNA binding domain of a transcription factor or to the activation domain of a transcription factor, respectively. Interaction between a compound of the library (representing the label) attached to the AGT fusion protein and the target protein Y leads to the formation of a functional transcription factor and gene expression only in the case where the compound in the chemical library, linked through the covalent AGT-substrate bond, to either the DNA binding domain of a transcription factor or to the activation domain of a transcription factor, binds to the target protein Y fused to the activation domain of a transcription factor or the DNA binding domain of a transcription factor, respectively. If gene expression is coupled to a selective advantage, those molecules of the library leading to the growth of the host can be identified.

In the case where L is a bond connecting R₄ to R₁ forming a cyclic substrate, a preferred compound is the cyclic substrate wherein the bond from R₄ to R₁ is a bond connecting the linker R₄ to an amino group R₆ as defined under formula 2. In such a preferred cyclic substrate R₂ is preferably an oligonucleotide, i.e. a β-D-2′-deoxyribosyl being incorporated into a single stranded oligodeoxyribonucleotide having a length of 2 to 99 nucleotides as detailed above. This oligonucleotide may be further chemically modified so that it can be detected and functions therefore as a label. The chemical modification of substituents might be of the same nature as mentioned above for the label L.

In the case where L is a further group —R₃—CH₂—X—R₁—R₂, the substrate is a dimeric compound leading to a dimerised fusion protein on reaction with a fusion protein comprising AGT.

### EXAMPLES

### Example 1

### Glutathion S-Transferase (C) hAGT Fusion Protein

hAGT is cloned between the BamH1 and EcoR1 sites of the expression vector pGEX2T (Pharmacia). Protein expression is carried out in E. coli strain JM83. An exponentially growing culture is induced with 1 mM IPTG and the expression is carried out for 3.5 h at 24° C. The harvested cells are resuspended in PBS supplemented with 1 mM PMSF and 2 μg/mL aprotinin and disrupted by lysozyme and sonification. To get rid of DNA, MgCl₂ is adjusted to 1 mM and DNAse I is added to a concentration of 0.01 mg/mL. The mixture is allowed to stand on ice for 30 min before cell debris are separated by centrifugation at 40000×g. The extract is applied to equilibrated glutathion sepharose which is then washed with one bed volume Tris.HCl pH 8.5 and with 20 bed volumes PBS. GST-hAGT fusion protein is then eluted with 10 mM reduced glutathione in 50 mM Tris.HCl pH 7.9. The purified protein is dialyzed against 50 mM HEPES pH 7.2; 1 mM DTT; 30% glycerol and then stored at −80° C. Purified GST-hAGT is incubated in vitro with O⁶-benzylguanine (Sigma) or O⁶-4-bromothenylguanine. In a total reaction volume of 90 μL, 0.4 μM GST-hAGT are incubated with 2 μM substrate in 50 mM HEPES pH 7.2; 1 mM DTT at room temperature. At several points of time an aliquot is quenched with 8.5 pmol O⁶-benzylguanineoligo-nucleotide which is linked to a biotin group via the O⁶ position (R. Damoiseaux et al., Chem Biochem 4: 285, 2001) for 10 min and mixed with SDS-Laemmli buffer for Western blotting analysis (neutravidin-peroxidase conjugate (PIERCE), Renaissance reagent plus (NEN)). The intensity of the corresponding bands is quantified by a Kodak Image Station 440.

### Example 2

### Orotidine-5′-phosphate decarboxylase Ura3 (C) hAGT Fusion Protein

A plasmid is used which is based on the yeast shuttle vector pRS314 (Sikorski and Hieter, Genetics 122: 19-27, 1999). Between the BamH1 and EcoR1 restriction sites of pRS314 a copper inducible promoter (CU-promoter) is inserted. The Ura3 gene (with an N terminal HA-tag) is inserted between the BgIII and KpnI sites, and hAGT is inserted between the EcoR1 and BgIII sites leading to a hAGT-Ura3 fusion protein.

Expression levels of the hAGT-Ura3 fusion protein are monitored by inducing 5 mL of a culture with an OD₆₀₀ of 0.3 with 0.1 mM CuSO₄ and incubating the culture for 3 h. 3 mL of the culture are harvested by centrifugation, resuspended in 50 μL 2× Laemmli buffer and disrupted by 3 freeze-thaw cycles. Samples are loaded to a SDS-PAGE and Western blotting is performed (mouse HA.11 antibody (BABCO); peroxidase conjugated anti mouse antibody A4416 (Sigma); Renaissance reagent plus (NEN)).

Activity of Ura3 is determined by growing transformants on plates containing CuSO₄ and lacking uracil. The activity of hAGT-Ura3 fusion protein is determined by an ELISA: 50 mL CM medium are supplemented with 0.1 mM CuSO₄ and 100 μM O⁶-benzylguanine, and inoculated with 5 mL of a stationary grown overnight culture. Protein expression is carried out for approximately 5 hours until the OD₆₀₀ reaches 1.0. The harvested cells are resuspended in yeast lysis buffer (50 mM HEPES pH 7.5; 150 mM NaCl; 5 mM EDTA; 1% TX100; 1 mM DTT; 1 mM PMSF; 2 μg/mL aprotinin) and disrupted by 3 freeze-thaw cycles. 300 μL of the resulting extract are incubated for 20 min with 5 pmol O⁶-benzylguanine- oligonucleotide which is linked to a biotin group via the O⁶ position (R. Damoiseaux et al., ChemBiochem 4: 285, 2001), and then coated for 1 h to a previously blocked StreptaWell plate (Boehringer Mannheim). The ELISA is then developed with standard methods (detection by HA.11 and A4416 antibodies; development with peroxidase substrate ABTS (1.0 mg/mL ABTS, 0,01% H₂O₂ in 100 mM sodium citrate); readout at 405 nm).

### Example 3

### Ubiquitin (N) Ura3 (C) hAGT Fusion Protein

To generate a hAGT with an N- terminal arginine a linear ubiquitin-hAGT fusion protein is constructed by PCR where the construct is flanked with EcoR1 and BgIII restriction sites. The construct is inserted between the EcoR1 and BgIII sites of the construct hAGT-Ura3 described in Example 2 leading to an ubiquitin-hAGT-Ura3 fusion protein.

Expression levels of the ubiquitin-hAGT-Ura3 fusion protein and activity of the fusion protein obtained is monitored as described for hAGT-Ura3 in Example 2.

### Example 4

### Tup1 (N)^{W160} hAGT Fusion Protein

Tup1 is involved in glucose repression of transcription (F. E. Williams and R. Trumbly, Mol Cell Biol 10: 6500-11, 1990). This nuclear localized protein is fused to the N-terminus of ^{W160}hAGT by the linker DHGSG, which contains the cloning site Nco I and connects the last amino acid Asn of Tup1 with the first amino acid Met of hAGT. For antibody detection the epitope HA is directly fused to the C-terminus of hAGT, followed by the stop codon. The primers for the cloning are ak121 (N, Tup1):

| | |
|---|---|
| ak121 (N, Tup1): | |
| 5′-GCATGAATTCATGACTGCCAGCGTTTCG-3′, | (SEQ ID No. 1) |
| ak122 (C, Tup1): | |
| 5′-GGATCCCCATGGTCATTTGGCGCTATTTTTTTA | (SEQ ID No. 2) |
| TAC-3′, | |
| ak125 (N, hAGT): | |
| 5′-CGTGACCATGGGAGTGGGATGGACAAGGATTGT | (SEQ ID No. 3) |
| GAAATG-3′ | |
| and | |
| ak132 (C, HA): | |
| 5′-GCATGGGTACCTTAAGCGTAATCTGGAACATC | (SEQ ID No. 4) |
| G-3′. | |

A culture of L40 yeast cells, containing the expression vector p314AK1 in which the Tup1-^{W160}hAGT protein is under control of the p_{cup1} promoter, is grown to an OD₆₀₀ of 0.6. Expression of Tup1-¹⁶⁰hAGT is induced by adding CuSO₄ to a concentration of 100 μM and the cell culture is incubated for 2.5 h. After lysis of the yeast cells by freeze/thaw cycling the cell extract is analyzed for the presence of expressed Tup1-^{W160}hAGT fusion protein using Western Blotting (1. antiHA-antibody (Babco), 2. antimouse-peroxidase conjugate (Sigma)). The activity is verified by fluorescence microscopy, when the nuclear fusion protein is labeled with BGAF (O⁶-(p-aminomethyl)benzylguanine carrying a diacetate of 5(6)-carboxy-fluorescein residue connected by an amide bond to the p-aminomethyl group) in vivo.

BGAF is prepared in the following way:

6.0 mg (0.022 mmol) of O⁶-(4-aminomethyl-benzyl)guanine are dissolved in 2 mL dry DMF (40° C., sonicated for 30 min) under argon atmosphere. After cooling to room temperature 4.6 μL triethylamine (0.033 mmol) and 14.8 mg (0.027 mmol) of 5(6)-carboxyfluorescein N-succinimidyl ester (mixture of isomers) are added. After stirring 1 h at room temperature the solvent is removed and the products are purified by flash column chromatography using a stepwise gradient of methanol in dichloromethane (1:20, 1:10, 1:5). Under these conditions both BGAF and the hydrolyzed derivative of BGAF (termed BGFL) are isolated, and are each dissolved in 400 μL DMSO. The concentration of the solution of BGFL is determined by the absorption at λ=492 nm via the extinction coefficient of fluorescein (ε₄₉₂=98.4×10³ M⁻¹cm⁻¹ at pH 7.4). The concentration of BGFL is calculated as 4.4 mM. Yield: 1.11 mg (0.0018 mmol, 8%). R_{f}=0.02 (methanol/dichloromethane 1/10). MS(ESI) 629.27 (100 [M+H]⁺). C₃₄H₂₄N₆O₇ M=628.61 g/mol. The concentration of BGAF is determined by the absorption at λ=280 nm using the added extinction coefficients of O⁶-(-4aminomethyl-benzyl)guanine and fluorescein (ε₂₈₀=(7.1+53.3) mM⁻¹ cm⁻¹=60.4 mM⁻¹ cm⁻¹). The concentration of BGAF is calculated as 0.8 mM. Yield: 0.23 mg (0.3 μmol, 1.5%). R_{f}=0.38 (methanol/dichloro-methane 1/10). MS(ESI) 713.35 (100 [M+H]⁺). C₃₈H₂₈N₆O₉ M=712.68 g/mol.

### Example 5

### Tup1 (N) Enhanced Cyano Fluorescent Protein ECFP (C) ^{W160}hAGT Fusion Protein

Tup1 is fused to the N-terminus of ^{W160}hAGT by the linker DHGSG as described in Example 4. However, the epitope HA fused to the C-terminus of hAGT is followed by the fluorescent protein ECFP. The primers for the cloning are ak121 (N, Tup1) (SEQ ID No. 1), ak122 (C, Tup1) (SEQ ID No. 2), ak125 (N, hAGT) (SEQ ID No. 3), ak126 (ECFP, HA):

| | |
|---|---|
| 5′-CTCGCCCTTGCTCACCATCCCGCTGCCGGACCC | (SEQ ID No. 5) |
| AGCGTAATCTGGAACATCG-3′, | |
| ak127 (ECFP, HA): | |
| 5′-CGATGTTCCAGATTACGCTGGGTCCGGCAGCGG | (SEQ ID No. 6) |
| GATGGTGAGCAAGGGCGAG-3′ | |
| and | |
| ak128 (C, ECFP): | |
| 5′-CTAGCTGGGTACCGTTACTTGTACAGCTCGTCC | (SEQ ID No. 7) |
| ATGA-3′. | |

A culture of L40 yeast cells, containing the expression vector p314AK1 in which the Tup 1-^{W160}hAGT-ECFP protein is under control of the p_{cup1} promoter, is grown to an OD₆₀₀ of 0.6. Expression of Tup 1-^{W160}hAGT-ECFP is induced by adding CuSO₄ to a concentration of 100 μM and the cell culture is incubated for 2.5 h. After lysis of the yeast cells by freeze/thaw cycling the cell extract is analyzed for the presence of expressed Tup 1-^{W160}hAGT-ECFP fusion protein using Western Blotting (1. antiHA-antibody (Babco), 2. antimouse-peroxidase conjugate (Sigma)). The activity is verified by fluorescence microscopy, when the nuclear fusion protein is labeled with BGAF in vivo and the nucleus is distinguished from the residual cell.

### Example 6

### LexA (C) hAGT Fusion Protein

LexA is the DNA-binding domain of an E. coli transcription regulator used in the yeast-two hybrid approach. The hAGT is fused to its C-terminus, in-between the restriction sites EcoR I and Not I of the yeast-expression vector pHybLexZeo (Invitrogen). The primers used are ak101 (N, hAGT):

| | |
|---|---|
| ak101 (N, hAGT): | |
| 5′-CGATACGAATTCATGGACAAGGATTGTGAAATG | (SEQ ID No. 8) |
| AAACGC-3′, | |
| and | |
| ak102 (C, hAGT): | |
| 5′-TTCATAGCGGCCGCGTCAGTTTCGGCCAGCAGG | (SEQ ID No. 9) |
| C-3′. | |

### Example 7

### Cytochrome C Peroxidase CCP (C) hAGT Fusion Protein

In the hAGT-Ura3 construct (Example 2) Ura3 is replaced by CCP (without its mitochondrial targeting sequence) carrying the mutations D217P and D224Y (Iffland et al., Biochem Biophys Res Commun 286: 126-132, 2001). To test the activity of CCP as a fusion protein, yeast colonies transformed with the vector leading to expression of hAGT-CCP are transferred to nitrocellulose and (after 3 freeze-thaw cycles) exposed to 5 or 20 mM ABTS in 50 mM KH₂PO₄ buffer containing 0.02% H₂O₂. The colonies stained dark green within minutes whereas colonies not expressing the protein only stained very faintly.

### Example 8

### Enhanced Cyano Fluorescent Protein ECFP (C) ^{W160}hAGT Fusion Protein

The fluorescent protein ECFP is fused to the C-terminus of ^{W160}hAGT, followed by a stop codon. The fusion by PCR is performed with the same primers as for the fusion protein Tup 1-^{W160}hAGT-ECFP (Example 5). The protein ^{W160}hAGT-ECFP is incorporated into the mammalian expression vector pNuc (Clontech) between the restriction sites Nhe I and BamH I.

CHO cells deficient in AGT are transfected with a vector encoding ^{W160}hAGT-ECFP. After 24 h of transient expression, cells grown on 0.18 mm thick glass slides are transferred to a perfusion chamber and incubated with BGFL (5 μM) for 5 min. Cells are washed three times with PBS buffer to remove excess substrate. For the fluorescence measurements a Zeiss LSM510 laser scanning confocal microscope is used (Card Zeiss AG). Detection of fluorescein or ECFP signals (excitation at 488 nm) is achieved by appropriate filters. Scanning speed and laser intensity are adjusted to avoid photobleaching of the fluorescent probes, and damage or morphological changes of the cells.

### Example 9

### Membrane Protein of the ER Sec62/DHFR (C) hAGT Fusion Protein

Fragments encoding the ORF (open reading frame) of the N-terminal domain of the protein Sec62p, the full-length ORF of the peroxisomal membrane proteins Pex10p and Pex15p, and the ORF of an N-terminal fragment of the yeast casein kinase (YCK1) are obtained by PCR using yeast genomic DNA as a template and an oligonucleotide primer complementary to the 5′ and 3′ ends of the desired DNA fragments respectively. All 5 -primers contain an additional BamHI site and all 3′-primers an additional restriction site to allow for the in-frame fusion 3′ to the CUP1-hAGT module on a pRS314 vector or for the DNA fragment of YCK1 on a pRS304 vector. The ORF of the N-terminal domain of the protein Sec62p is inserted in frame between the CUP1-hAGT module and the sequence encoding the mouse dihydrofolate reductase (DHFR) that is extended by an additional sequence encoding for the HA epitope tag (Dha). The CUP1-hAGT module is obtained by PCR using a plasmid DNA containing the full length AGT as a template and an oligonucleotide primer complementary to the 5′ and 3′ ends of the ORF of hAGT. The 3′-primers contain an additional BamHI site and the 5′-primer an additional EcoRI site to allow for the fusion 3′ to the yeast CUP1 promotor on a pRS314 and pRS304 vector. The plasmids CUP1-hAGT-SEC62-314, CUP1-hAGT-PEX10-314 and CUP1-hAGT-PEX15-314 are transformed into yeast cells. The presence of the plasmids are controlled by the growth on selective media lacking tryptophan. To obtain the full length version of the hAGT-YCK1 fusion gene, CUP1-hAGT-YCK1-304 is cut with Sal1 to allow for homologous recombination with the chromosomal YCK1 after transformation of the cut plasmid into yeast. Successful recombination is verified by diagnostic PCR using the appropriate oligonucleotides as primers.

Functional assay of the hAGT-Sec62-Dha fusion protein: 100 mL of S. cerevisiae cells expressing hAGT-Sec62-Dha are grown at 30° C. to an OD₆₀₀ of ˜0.5 and supplemented with 100 μM CuSO₄ 4 hours prior to cell extraction. After centrifugation the cells are opened by grinding in fluid nitrogen and the proteins are extracted in buffer containing 150 Mm NaCl, 20 mM HEPES pH 7.5, 1 mM EDTA and a protease inhibitor cocktail (Boehringer Mannheim, Germany). After a 15 min centrifugation at 20.000 rpm at 4° C., the cleared extracts are treated with 10 pmol of an oligonucleotide containing the substrate BGBT for 20 min at room temperature. The cells extracts are incubated with 15 μL of Dynabeads for 4 hours and the beads are washed five times with 1 ml of extraction buffer. The washed beads are boiled in 30 μL of Laemmli buffer and the extract is subjected to SDS PAGE. The purified hAGT-Sec62-Dha is detected after Western blotting onto nitrocellulose by consecutive incubation with mouse monoclonal HA antibody and horseradish peroxidase-coupled rabbit anti-mouse antibody.

### Example 16

### Serotonin Receptor 5-HT₃ (N) hAGT Fusion Protein

The vector pEAK8-5HT₃R containing the serotonin receptor 5-HT₃ (mouse) was provided by the group of H. Vogel (EPFL Lausanne, Switzerland). ^{W160}hAGT is incorporated into the fourth loop (cytoplasmatic) of the receptor between the restriction sites SnaB I and Pac I, which had been introduced by mutagenesis. The primers for the amplification of the ^{W160}hAGT are ak144 (N, ^{W160}hAGT):

| | |
|---|---|
| ak144 (N, ^{W160}hAGT): | |
| 5′-GCATGCTACGTAATGGACAAGGATTGTGAAA | (SEQ ID No. 10) |
| TG-3′, | |
| ak145 (C, ^{W160}hAGT): | |
| 5′-GAGCACTTAATTAAGTTTCGGCCAGCAGGCG | (SEQ ID No. 11) |
| G-3′. | |

CHO cells deficient in AGT are transfected with a vector encoding 5-HT₃-(^{W160}hAGT) ^{loop4}-receptor. After 24 h of transient expression, cells grown on 0.18 mm thick glass slides are transferred to a perfusion chamber and incubated with BGFL (5 μM) for 5 min. Cells are washed three times with PBS buffer to remove excess substrate. For the fluorescence measurements a Zeiss LSM510 laser scanning confocal microscope is used (Carl Zeiss AG). Detection of fluorescein signals (excitation at 488 nm) is achieved by appropriate filters. Scanning speed and laser intensity are adjusted to avoid photobleaching of the fluorescent probes, and damage or morphological changes of the cells.

### Example 11

### Human Estrogen Receptor hER (C) hAGT Fusion Protein

The vector pC1-hER containing the human estrogen receptor was provided by the group of H. Vogel (EPFL, Lausanne, Switzerland). ^{W160}hAGT is fused to the C-terminus of the receptor between the restriction sites Nhe I and Xho I The primers for the amplification of the ^{W160}hAGT are ak136 (N, ^{W160}hAGT):

| | |
|---|---|
| ak136 (N, ^{W160}hAGT): | |
| 5′-ATCGAGCTAGCGCTACCGGTCGCCACCATGG | (SEQ ID No. 12) |
| ACAAGGATTGTGAAATG-3′ | |
| and | |
| ak151 (C, ^{W160}hAGT): | |
| 5′-CGTAGCTCGAGAGTTTCGGCCAGCAGG | (SEQ ID No. 13) |
| C-3′. | |

CHO cells deficient in AGT are transfected with a vector encoding ^{W160}hAGT-hER. After 24 h of transient expression, cells grown on 0.18 mm thick glass slides are transferred to a perfusion chamber and incubated with BGFL (5 μM) for 5 min. Cells are washed three times with PBS buffer to remove excess substrate. For the fluorescence measurements a Zeiss LSM510 laser scanning confocal microscope is used (Carl Zeiss AG). Detection of fluorescein signals (excitation at 488 nm) is achieved by appropriate filters. Scanning speed and laser intensity are adjusted to avoid photobleaching of the fluorescent probes, and damage or morphological changes of the cells. The labeling of the fusion protein ^{W160}hAGT-hER located in the nucleus is verified. The nucleus is clearly distinguishable from the rest of the cell.

### Example 12

### SV40 Large T Antigen Nuclear Localization Sequence NLS (C) hAGT and NLS/ECFP (C) hAGT

The three copies of the nuclear localization signal (NLS₃) of the simian virus 40 large T-antigen are either fused at the C-terminus of the fluorescent protein ECFP fused to a HA-tag fused to the C-terminus of ^{W160}hAGT yielding ^{W160}hAGT-HA-ECFP-NLS₃, or are fused directly to the C-terminus of ^{W160}hAGT yielding ^{W160}hAGT-NLS₃. The fusion by PCR is performed with the same primers as for the fusion protein Tup 1-^{W160}hAGT-ECFP (Example 5). Then ^{W160}hAGT-HA-ECFP-NLS₃ or ^{W160}hAGT-NLS₃, respectively, are incorporated into the mammalian expression vector pNuc (Clontech) between the restriction sites Nhe I and BgI II. The primers are ak136 (N, ^{W160}hAGT) (SEQ ID No. 12), ak137 (C, ECFP):

| | |
|---|---|
| 5′-CATGCAGATCTGAGTCCGGACTTGTACAGCT | (SEQ ID No. 14) |
| C-3′ | |
| and | |
| ak107 | |
| (C, ^{W160}hAGT): | |
| 5′-CCAGGCAGATCTGTTTCGGCCAGCAGGCGGG | (SEQ ID No. 15) |
| G-3′. | |

CHO cells deficient in AGT are transfected with the vector pNuc encoding ^{W160}hAGT-HA-ECFP-NLS₃ or alternatively ^{W160}hAGT-NLS₃. After 24 h of transient expression, cells grown on 0.18 mm thick glass slides are transferred to a perfusion chamber and incubated with BGFL (5 μM) for 5 min. Cells are washed three times with PBS buffer to remove excess substrate. For the fluorescence measurements a Zeiss LSM510 laser scanning confocal microscope is used (Carl Zeiss AG). Detection of fluorescein or ECFP signals (excitation at 488 nm) is achieved-by appropriate filters. Scanning speed and laser intensity are adjusted to avoid photobleaching of the fluorescent probes, and damage or morphological changes of the cells.

1. A labelled AGT fusion protein comprising a protein of interest selected from the group consisting of enzymes, DNA-binding proteins, transcription regulating proteins, membrane proteins, nuclear receptor proteins, nuclear localization signal proteins, protein cofactors, small monomeric GTPases, ATP-binding cassette proteins, intracellular structural proteins, proteins with sequences responsible for targeting proteins to particular cellular compartments, proteins generally used as labels or affinity tags, and domains or subdomains of the aforementioned proteins, with the proviso that the major head protein D of phage λ (gpD), and the proteins MHHHHHHSSA, DHFR-HA, V5-NLS-B42, HA-Ura3 and SSN6 are excluded.

2. The labelled AGT fusion protein according to claim 1 wherein the protein of interest is a membrane protein.

3. The labelled AGT fusion protein according to claim 1 wherein the protein of interest is a kinase.

4. The labelled AGT fusion protein according to claim 1 wherein the protein of interest is a nuclear receptor protein.

5. The labelled AGT fusion protein according to claim 1 wherein the protein of interest is a phosphatase.

6. The labelled AGT fusion protein according to claim 1 wherein the protein of interest is a protease.

7. The labelled AGT fusion protein according to claim 1 which consists of one or more proteins of interest fused to AGT at the N—, C— or N— and C-terminal of AGT and a substrate carrying a label.

8. The labelled AGT fusion protein according to claim 1 wherein AGT is a variant of human AGT with one or more amino acid substitution, deletion or addition.

9. The labelled AGT fusion protein according to claim 8 wherein AGT is a variant wherein Asn¹⁵⁷ is replaced by Gly and Ser¹⁵⁹ by Glu, and the one wherein Gly¹⁶⁰ is replaced by Ala or Trp.

10. The labelled AGT fusion protein according to claim 8 wherein AGT is a variant wherein Asn¹⁵⁷ is replaced by Ser, Ser¹⁵⁹ by His, and Gly¹⁶⁰ by Asn.

11. The labelled AGT fusion protein according to claim 1 wherein the label is a spectroscopic probe; a radioactively labelled molecule; a molecule which is one part of a specific binding pair which is capable of specifically binding to a partner; a molecule that is suspected to interact with other biomolecules; a library of molecules that are suspected to interact with other biomolecules; a molecule which is capable of crosslinking to other molecules; a molecule which is capable of generating hydroxyl radicals upon exposure to H₂O₂ and ascorbate; a molecule which is capable of generating reactive radicals upon irradiation with light; a molecule covalently attached to a solid support; a nucleic acid or a derivative thereof capable of undergoing base-pairing with its complementary strand; a lipid or other hydrophobic molecule with membrane-inserting properties; a biomolecule with desirable enzymatic, chemical or physical properties; or a molecule possessing a combination of any of the properties listed above.

12. The labelled AGT fusion protein according to claim 11 wherein the label is a fluorophore, a chromophore, a magnetic probe or a contrast reagent.

13. The labelled AGT fusion protein according to claim 12 wherein the label is a fluorophore.

14. The labelled AGT fusion protein according to claim 11 wherein the label is a molecule which is one part of a specific binding pair which is capable of specifically binding to a partner.

15. The labelled AGT fusion protein according to claim 11 wherein the label is a molecule which is capable of crosslinking to other molecules.

16. The labelled AGT fusion protein according to claim 11 wherein the label is a molecule attached to a solid support.

17. The labelled AGT fusion protein according to claim 16 wherein the solid support is a chemically modified oxidic surface, glass surface, polymer surface, functionalised polymer, noble metal surface.

18. The labelled AGT fusion protein according to claim 17 wherein the solid support is in the form of a bead, microtiter plate or sensor element.

19. The labelled AGT fusion protein according to claim 11 wherein the label is a nucleic acid or a derivative thereof capable of undergoing base-pairing with its complementary strand.

20. The labelled AGT fusion protein according to claim 1 comprising a plurality of labels.

21. An AGT fusion protein comprising a protein of interest selected from the group consisting of enzymes, DNA-binding proteins, transcription regulating proteins, membrane proteins, nuclear receptor proteins, nuclear localization signal proteins, protein cofactors, small monomeric GTPases, ATP-binding cassette proteins, intracellular structural proteins, proteins with sequences responsible for targeting proteins to particular cellular compartments, proteins generally used as labels or affinity tags, and domains or subdomains of the aforementioned proteins, with the proviso that the major head protein D of phage λ (gpD), and the proteins MHHHHHHSSA, DHFR-HA, V5-NLS-B42, HA-Ura3 and SSN6 are excluded.

22. The AGT fusion protein according to claim 21 wherein the protein of interest is a membrane protein.

23. The AGT fusion protein according to claim 21 wherein the protein of interest is a kinase.

24. The AGT fusion protein according to claim 21 wherein the protein of interest is a nuclear receptor protein.

25. The AGT fusion protein according to claim 21 wherein the protein of interest is a phosphatase.

26. The AGT fusion protein according to claim 21 wherein the protein of interest is a protease.

27. The AGT fusion protein according to claim 21 which consists of one or more proteins of interest fused to AGT at the N—, C— or N— and C-terminal of AGT and a substrate carrying a label.

28. The AGT fusion protein according to claim 21 wherein AGT is a variant of human AGT with one or more amino acid substitution, deletion or addition.

29. The AGT fusion protein according to claim 28 wherein AGT is a variant wherein Asn¹⁵⁷ is replaced by Gly and Ser¹⁵⁹ by Glu, and the one wherein Gly¹⁶⁰ is replaced by Ala or Trp.

30. The AGT fusion protein according to claim 28 wherein AGT is a variant wherein Asn¹⁵⁷ is replaced by Ser, Ser¹⁵⁹ by His, and Gly¹⁶⁰ by Asn.

31. A variant of human AGT wherein Asn¹⁵⁷ is replaced by Gly and Ser¹⁵⁹ by Glu, or wherein Gly¹⁶⁰ is replaced by Ala or Trp, or wherein Asn¹⁵⁷ is replaced by Ser, Ser¹⁵⁹ by His, and Gly¹⁶⁰ by Asn.

32. A method for detecting and manipulating a protein of interest, characterized in that the protein of interest incorporated into an AGT fusion protein is contacted with a suitable AGT substrate carrying a label, and the AGT fusion protein is detected and optionally further manipulated using the label in a system designed for recognising or handling the label.

33. The method according to claim 32 further comprising the step of forming an AGT fusion protein from the protein of interest and AGT.

34. The method according to claim 32 wherein the protein of interest is selected from the group consisting of enzymes, DNA-binding proteins, transcription regulating proteins, membrane proteins, nuclear receptor proteins, nuclear localization signal proteins, protein cofactors, small monomeric GTPases, ATP-binding cassette proteins, intracellular structural proteins, proteins with sequences responsible for targeting proteins to particular cellular compartments, proteins generally used as labels or affinity tags, and domains or subdomains of the aforementioned proteins, with the proviso that the major head protein D of phage λ (gpD), and the proteins MHHHHHHSSA, DHFR-HA, V5-NLS-B42, HA-Ura3 and SSN6 are excluded.

35. The method according to claim 34 wherein the protein of interest is a membrane protein.

36. The method according to claim 34 wherein the protein of interest is a kinase.

37. The method according to claim 34 wherein the protein of interest is a nuclear receptor protein.

38. The method according to claim 34 wherein the protein of interest is a phosphatase.

39. The method according to claim 34 wherein the protein of interest is a protease.

40. The method according to claim 32 wherein the AGT fusion protein consists of one or more proteins of interest fused to AGT at the N—, C— or N— and C-terminal of AGT.

41. The method according to claim 32 wherein AGT in the AGT fusion protein is human AGT or a variant of human AGT with one or more amino acid substitution, deletion or addition.
